(19) United States
(12) Patent Application Publication
(10) Pub. No.: US 2022/0213471 A1
(43) Pub. Date: Jul. 7, 2022

(54) SMALL MOLECULE SCREENING CELLULAR ASSAY USING MODIFIED BEADS

(71) Applicant: HOFFMANN-LA ROCHE INC., Little Falls, NJ (US)

(72) Inventors: Alexander Lee SATZ, Basel (CH); Gregor DERNICK, Basel (CH); Claudio ZAMBALDO, Basel (CH)

(21) Appl. No.: 17/594,367

(22) PCT Filed: Apr. 15, 2020
(86) PCT No.: PCT/EP2020/060616

§ 371 Date: Oct. 13, 2021

(30) Foreign Application Priority Data
Apr. 16, 2019 (EP) 19169563.4

(51) Int. Cl. C12N 15/10; G01N 33/50; G01N 33/58

(52) U.S. Cl. CPC C12N 15/1075; G01N 33/582; G01N 33/5023; G01N 33/5014

(57) ABSTRACT

A method for screening a DNA-encoded library of chemical structures (2) for activity in a cellular target (11) wherein the chemical structures (2) of the library, the corresponding encoding DNA (4) and, optionally, a chemical probe (7/8/9) susceptible to the response molecule (12) are covalently linked to beads (1); the method comprising providing an incubation medium (13) or aliquot thereof comprising the cellular target (11) and exactly one or more than one bead (1) as defined above, releasing the chemical structures (2) from the bead(s) (1) in the incubation medium (13) or aliquots thereof by cleaving the structure linkers (3) and incubating the released chemical structures (2) and the cellular target (11); and sequencing the encoding DNA present or remaining on the bead(s) (1). A bead (1) suited for the method is also provided.

The present invention concerns a screening assay for screening small molecules for potential efficacy in altering the activity of a pharmaceutically interesting cellular target, and a means for carrying out the assay.

### BACKGROUND

Most targets of pharmaceutical interest are active in a cellular context. When investigating small molecules altering target activity, it may be beneficial to do this in a cellular context, including when screening of large diversities of compounds.

One of the desired reactions of the system to a target-modulating small molecule could be the release of molecules from the cell. These molecules could be enzymes, proteins, nucleic acids or smaller products of the cell such as metabolites. The release could occur by directed release or by simple leakage. During a screen, those released molecules would be detected as a means of compound action.

Screening of small-molecule compounds in cellular assays in the pharmaceutical industry is generally accomplished by combining a compound and cells in a single container (Jones E, Michael S, Sittampalam G S. Basics of Assay Equipment and Instrumentation for High Throughput Screening. 2012 May 1 [Updated 2016 Apr. 2]. In: Sittampalam G S, Coussens N P, Brimacombe K, et al., editors. Assay Guidance Manual [Internet]. Bethesda (Md.): Eli Lilly & Company and the National Center for Advancing Translational Sciences; 2004). The small molecule can thus either act on the cellular molecules on the outside of the cell or in the cellular membrane, or it could enter the cell and execute its action inside the cell. The ability of the compound to modulate the activity of the cell is then determined by monitoring a feasible assay readout. This assay readout could be the processing of a substrate by an enzyme for instance. Processing of substrate may generate a fluorescent signal. Another means is, for example, where binding of a small molecule to the protein target is monitored, e.g. by change of fluorescence.

Large numbers of different small molecules may be interrogated (=screened) for their ability to modulate target activity or binding by using microcompartments, such as in the form of n-well plates (where n is commonly 96, 384, or 1536). Commercially available microplate readers are commonly used to measure the activity of each small molecule via measurement of remaining substrate (or appearance of product) for each individual well in the plate. Another known way of splitting up such an assay into a multiplicity of microcompartments is the generation of water droplets containing the assay reagents in the form of a water-in-oil emulsion.

When employing a microplate reader, the logistics of long-term storage of small molecules plates is challenging, particularly when handling a very large number of plates. Additionally, the time required to assess product formation in microplates increases linearly with the number of small molecules, and this is problematic when investigating millions of small molecules (using current techniques, assessing 2 million molecules takes ˜10 business days) (Brouzes E., Medkova M., Savenelli N., Marran D., Twardowski M., Hutchison J. B., Rothberg J. M, Link D. R., Perrimon N., Samuels. M. L., PNAS 106, pp. 14195-14200 (2009)).

Any such assay requires that the structure of the chemical compounds found useful be readily determined. A solution to this problem is the provision of the compounds to be screened in the form of a DNA encoded chemical library (DELs) (Brenner and Lerner, Proc. Natl. Acad. Sci. USA. 89: 5381-5383 (1992)). In a DEL, each compound is beforehand linked (tagged) with a unique DNA sequence which corresponds to its structure. Thus, instead of needing to identify the structure of the useful compounds themselves (which task may depend on the structure and may even be impossible), only the corresponding DNA tag needs to be sequenced, which is a standard procedure identical for all useful compounds.

However, such DEL-based assay lags from the problem that the compounds associated with the DNA tag may not behave identically in the assay as the free compound. In WO 2018/087539 it is suggested to cleave before the assay the compound and its associated DNA tag, but to leave the free compound and its associated DNA tag in “spatial association” with each other.

ACS Chem. Biol. 13, pp. 761-771 (2018) discloses a small molecule assay using silica beads with quenched fluorophore probes and small molecules attached thereto and having DNA tags. In this assay the beads penetrate into cellular targets which thus by themselves act as microcompartments. The small molecules are cleaved off the penetrated beads by light. Any small molecule lethal to the cellular target cause apoptosis thereof, release of caspase-3 and, in the still penetrated bead(s), the removal of the quencher from the fluorophore. Such now fluorescent cellular targets (thus now fluorescent microcompartments) are sorted out using flow cytometry and the DNA of any beads comprised therein is then sequenced to find out the corresponding small molecules that caused apoptosis.

U.S. Pat. No. 5,958,703 A discloses a support with a tether which is modifiable by a reporter molecule, and associated screening processe. This publication “isolates” supports having modified tethers. This publication does therefore not pool all supports before isolating supports with modified tethers.

WO 2013/057188 A1 releases compounds such that they “remain inside the solid supports/beads” or “that each compound is physically located inside its parent solid support” or is released “inside” the beads”, or even that a “substrate” is allowed “to be absorbed into” the supports/beads. This publication does thus not release compounds to be assayed into an incubation medium but retains them inside the beads. Accordingly the “physicochemical or biological system” that this publication targets are soluble species, but not cellular targets: In the latter case there would be two heterogenous phases (the compound-containing beads and the cellular targets) which would preclude interaction between them.

ACS Comb. Sci. 19, pp. 524-532 (2017) describes the principle of a water-in-oil droplet-based assay. The beads of a DNA-encoded library are encapsulated in the droplets and incubated in the test assay. Any droplets showing positive reaction in the incubation assay (thus any positive microcompartments) are first sorted out, and the beads isolated from such sorted out droplets are then further examined for being a statistically relevant hit.

ACS Comb. Sci. 21, pp. 425-435 (2019) also describes a water-in-oil droplet-based assay. The beads of a DNA-encoded library are encapsulated in the droplets and incubated in a screening assay for autotaxin inhibiting activity. This assay does not use a cellular target but homogeneously dissolved autotaxin as the target. This publication also first sorts out hit droplets: FIG. 1 shows the “droplet sorting junction (5)” using fluorescence detection.

It emerges that in these prior art bead-based assays the “positive” microcompartments (such as microwells, droplets, cells or other) were first selected or sorted out, then all beads contained in the sorted out microcompartments were pooled and their DNA tags analysed. The “positive” microcompartments however possibly contained more than one bead, of which normally only one is a hit bead (i.e. a bead which provides an active small molecule) and the other ones are non-hit beads. The foregoing publication identified these non-hit beads as “passenger beads” and derived a mathematical expression for a “false discovery rate” describing the extent of isolation and discovery of such non-hit beads. The confirmation that an isolated bead is a non-hit bead is only possible in a 2^{nd} round of testing.

The probability that in such prior art assays an isolated bead was a hit bead, Pₕ, can be calculated, as explained in the following.

The bead population, P_{b}, in the microcompartments is Poisson-distributed:

$${P}_{b} = \frac{{λ}^{{k}_{m}} {e}^{- λ}}{[ {k}_{m} ] !} \qquad ( 1 )$$

wherein kₘ is the number of beads in the microcompartment in question (thus 0, 1, 2). The kₘ cannot exceed an upper threshold value K that depends on the volume of the beads and on the volume of the microcompartments and on the reproducibility of that volume. It is e.g. impossible that the total volume of all beads contained in a microcompartment should be larger than the volume of the microcompartment itself. If the volume of the microcompartments was variable and could go up to infinity, then K could also go towards infinity. Real microcompartments, such as microwells or droplets, however are quite small and quite well reproducible in volume. So for real microcompartments the K will typically be less than 10. Reducing the volume of the microcompartments and thus K is also one customary way of lowering λ: The average bead population λ of the microcompartments must be smaller than their maximum possible bead population K. The λ is the average bead population of the microcompartments, defined as

$$λ = \frac{{{\sum }^{M}}_{i = 1} {( {k}_{m} )}_{i}}{M} \qquad ( 2 )$$

wherein M is the number of all microcompartments and the sum runs over all M microcompartments. Said P_{b} is the probability that a given microcompartment actually does contain (kₘ) beads. Bead distributions of this type are obtained with all known equipments for splitting up a bead population into microcompartments.

The probability Pₚ to have within said kₘ beads of a given microcompartment exactly kₚ hit beads is hypergeometrically distributed:

$${P}_{p} = \frac{( \begin{matrix}r ɛ N \\ {k}_{p}\end{matrix} ) ( \begin{matrix}ɛ ( 1 - r ) N \\ {k}_{m} - {k}_{p}\end{matrix} )}{( \begin{matrix}ɛ N \\ {k}_{m}\end{matrix} )} \qquad ( 4 )$$

wherein N is the number of individual chemical structures in the DNA-encoded library, r is the so-called “library hit rate” (the fraction of library compounds that are “active” chemical structures in the assay in question; a near zero but unknown number), ε is the so-called “library equivalent” (the number of beads that have one and the same chemical structure of the library linkend thereto), kₚ is the number of hit beads, and kₘ is as defined above. The assumption is here that εN, εN and ε(1−r)N are integer numbers, or are rounded to integer numbers.

The probability P that a given microcompartment contains kₘ beads and wherein exactly kₚ beads are, after incubation with the cellular target, hit beads (these events are independent from each other) is:

$$P = {P}_{b} \times {P}_{p} = \frac{{λ}^{{k}_{m}} {e}^{- λ}}{[ {k}_{m} ] !} \frac{( \begin{matrix}r ɛ N \\ {k}_{p}\end{matrix} ) ( \begin{matrix}ɛ ( 1 - r ) N \\ {k}_{m} - {k}_{p}\end{matrix} )}{( \begin{matrix}ɛ N \\ {k}_{m}\end{matrix} )} = {λ}^{{k}_{m}} {e}^{- λ} [ ɛ N - {k}_{m} ] ! \frac{( \begin{matrix}r ɛ N \\ {k}_{p}\end{matrix} ) ( \begin{matrix}ɛ ( 1 - r ) N \\ {k}_{m} - {k}_{p}\end{matrix} )}{[ ɛ N ] !} \qquad ( 5 )$$

The prior art processes recognized an incubated microcompartment as “positive” if its associated kₚ is greater than 0 (thus contained at least one hit bead). Such “positive” microcompartment then also had a kₘ greater than 0. The amount of all beads (hits and non-hits) obtained from all such “positive” microcompartments, Bₜₒₜ, is thus

$${B}_{tot} = M {\sum }_{{k}_{m} = 1}^{K} [ {k}_{m} {\sum }_{{k}_{p} = 0}^{{k}_{m}} ( {λ}^{{k}_{m}} {e}^{- λ} [ ɛ N - {k}_{m} ] ! \frac{( \begin{matrix}r ɛ N \\ {k}_{p}\end{matrix} ) ( \begin{matrix}ɛ ( 1 - r ) N \\ {k}_{m} - {k}_{p}\end{matrix} )}{[ ɛ N ] !} ) ] \qquad ( 6 )$$

wherein the sum in kₚ runs over all possible numbers of hit beads in the microcompartment, up to the total number of beads kₘ therein, and the sum in kₘ runs up to the upper threshold value K, which is the maximum number of beads that a microcompartment can contain. Formula (6) assumes that a given microcompartment contains either one number kₘ or another number kₘ′ of beads are mutually exclusive events, and that a given microcompartment with given number kₘ of beads contains either one number kₚ or another number kₚ of hit beads are also mutually exclusive events.

The amount of hit beads that is obtained from all such “positive” microcompartments, Bₕ, is

$${B}_{h} = M {\sum }_{{k}_{m} = 1}^{K} [ {\sum }_{{k}_{p} = 1}^{{k}_{m}} ( {k}_{P} {λ}^{{k}_{m}} {e}^{- λ} [ ɛ N - {k}_{m} ] ! \frac{( \begin{matrix}r ɛ N \\ {k}_{p}\end{matrix} ) ( \begin{matrix}ɛ ( 1 - r ) N \\ {k}_{m} - {k}_{p}\end{matrix} )}{[ ɛ N ] !} ) ] \qquad ( 7 )$$

wherein all symbols and explanations are as above. Formula (7) makes the same assumptions concerning mutual exclusivity as explained above for formula (6).

The abovementioned Pₕ is then

$$( 8 )$$

$${P}_{h} = \frac{{B}_{h}}{{B}_{tot}} = \frac{( {\sum }_{{k}_{m} - 1}^{K} [ \begin{matrix}{λ}^{{k}_{m}} [ ɛ N - {k}_{m} ] ! {\sum }_{{k}_{p} = 1}^{{k}_{m}} \\ ( \frac{{k}_{p}}{[ r ɛ N - {k}_{p} ] ! [ {k}_{p} ] ! [ ɛ ( 1 - r ) N - ( {k}_{m} - {k}_{p} ) ] ! [ {k}_{m} - {k}_{p} ] !} )\end{matrix} ] )}{( {\sum }_{{k}_{m} - 1}^{K} [ \begin{matrix}{λ}^{{k}_{m}} [ ɛ N - {k}_{m} ] ! {\sum }_{{k}_{p} = 0}^{{k}_{m}} \\ ( \frac{{k}_{m}}{[ r ɛ N - {k}_{p} ] ! [ {k}_{p} ] ! [ ɛ ( 1 - r ) N - ( {k}_{m} - {k}_{p} ) ] ! [ {k}_{m} - {k}_{p} ] !} )\end{matrix} ] )}$$

This Pₕ is smaller than 1 because the kₚ's in the numerator are smaller than the corresponding kₘ in the denominator. Pₕ becomes smaller with increasing λ because the λ polynomial in the denominator increases faster with increasing λ than the λ polynomial in the numerator, again because the kₚ's are smaller than the corresponding kₘ. Pₕ however has a maximum at λ=0. That is, these prior art processes have the lowest extent of false discovery of non-hit beads at a practically unfeasible average bead population λ of the microcompartments. A practically feasible average bead population has λ greater than 0 (there should be microcompartments having one or more beads contained therein).

There is thus no optimum in the prior art processes for both minimal extent of false discovery and analysis of non-hit beads and practically feasible bead population of microcompartments; the one must be traded in for the other.

The present invention seeks to overcome the foregoing problems.

### SUMMARY OF THE INVENTION

The invention provides:

1. A method for screening a DNA-encoded library of chemical structures for activity in a cellular target; wherein the cellular target is known to either release, or alter the release, of a response molecule when being contacted with an active chemical structure; wherein the chemical structures of the library, the corresponding encoding DNA and, optionally, a chemical probe susceptible to the response molecule are covalently linked to beads wherein each bead comprises
a) multiple instances of one sole chemical structure of the library, each instance being covalently linked over a structure linker to the bead, the structure linker being cleavable at a cleavable structure linker site; and
b) multiple instances of a DNA sequence encoding for that chemical structure, each DNA sequence being covalently linked over a tag linker to the bead, the tag linker comprising a cleavable tag linker site and being cleavable by a cleaving agent;
c) the cleavable structure linker site is not cleavable under reaction conditions that cleave the cleavable tag linker site, and vice versa;
and the tag linkers and/or the cleavable tag linker sites and/or the DNA sequences are optionally cleavable by the response molecule; provided that, if the encoding DNA sequences and/or the cleavable tag linker sites and/or the tag linkers are cleavable by the response molecule, then the bead is preferably devoid of the response molecule-susceptible chemical probe;
   the method comprising the steps of:
   (i) either
(i-a) providing, for each individual chemical structure and each individual cellular target to be assayed, an incubation medium comprising the cellular target and one or more bead(s) as defined above, the bead(s) having that individual chemical structure linked thereto, releasing the chemical structures from the bead(s) in the incubation medium by cleaving the structure linkers at the cleavable structure linker site, and incubating the cellular target and the released chemical structures in the incubation medium;
   or
(i-b) providing one sole incubation medium comprising the cellular target and all beads as defined above, having all chemical structures of the library linked thereto, separating from the incubation medium aliquots thereof comprising one or more beads, releasing in each of the aliquots the chemical structures from the contained bead by cleaving the structure linkers at the cleavable structure linker site, and incubating the cellular target and the released chemical structures in the aliquots of incubation medium;
   (ii) either, if the encoding DNA sequences and/or the cleavable tag linker sites and/or the tag linkers are cleavable by the response molecule:
(ii-a-1) the incubation media or the aliquots thereof are monitored for release of any encoding DNA sequences or fragments thereof from any beads; and if so, all beads are isolated from all incubation media or from all aliquots thereof, and all isolated beads are pooled;
(ii-a-2) the cleavable tag linker sites in the pooled beads are cleaved by the cleaving agent to release any encoding DNA sequences or fragments thereof;
(ii-a-3) the released encoding DNA sequences or fragments thereof are amplified and sequenced, to identify among them any complete DNA sequences of the DNA encoded library; and
(ii-a-4) the remainder of the complete DNA sequences of the DNA encoded library that were not identified in step (ii-a-3) are correlated with the corresponding chemical structures of the the DNA encoded library;
   or alternatively, if the bead(s) comprise(s) the response molecule-susceptible chemical probe:
(ii-b-1) the incubation media or aliquots thereof are monitored for any reaction, or change of reaction, of any probe(s) to the response molecule, and if so, all beads are isolated from all incubation media or from all aliquots thereof and are pooled;
(ii-b-2) beads showing said probe reaction, or said change of probe reaction, are extracted from the pool;
(ii-b-3) the cleavable tag linker sites in the beads extracted from the pool are cleaved by the cleaving agent to release any DNA sequences covalently linked to the isolated beads;
(ii-b-4) the released DNA sequences are amplified and sequenced; and
(ii-b-5) any DNA sequences sequenced in step (ii-b-3) are correlated with corresponding chemical structure(s) of the the DNA encoded library;
   and
   (iii) any chemical structure so correlated in step (ii-a-4) or (ii-b-5) is selected as a further said active chemical structure.

Preferred embodiments of the process are according to the dependent claims.

### DESCRIPTION OF THE FIGURES

FIG. 1 is a schematic representation of a bead suitable for the process of the invention, forming itself part of the invention.

FIG. 2 is a schematic representation of another bead suitable for the process of the invention.

FIG. 3 is a schematic representation of an incubation medium containing the bead and the cellular target.

### DETAILED DESCRIPTION OF THE INVENTION

In contrast to the prior art processes the inventive process first isolates and pools all beads (whether hit or non-hit beads) from all incubation media or aliquots thereof from all assay compartments (whether “positive” or not), then identifies hit beads in the pooled beads. In the inventive process the Pₕ for the sorted out beads is unity and is independent from the average bead population λ that was present in the microcompartments. It does not matter that in the instant process more beads must be identified based on probe reaction, or based on DNA sequencing: For the former there are very powerful sorting automated sorting tools (such as, in case of fluorescent probes the FACS), and for the latter there is e.g. automated next generation sequencing (NGS). Furthermore there is the possibility to examine only a part of the bead pool for hit beads. Such a part of the bead pool may be a representative fraction thereof, e.g. containing a number of beads which is the number of chemical structures present in the library multiplied by the “library equivalent” ε defined in the introduction, wherein c may typically be in the range of 1 to 100, preferably 5 to 50.

One advantageous effect of first pooling all beads, then sorting out the hit beads is explained in the following four paragraphs.

The processes of the prior art mentioned in in the introduction first sort out hit microcompartments (such as hit droplets). Therefore all microcompartments, bead-containing or not, need to be examined for activity by the sorter. If it is envisaged to obtain Bₕ hit beads then the number of necessary microcompartments, Mₚᵣᵢₒᵣₐᵣₜ, that in the prior art processes must be examined for activity is, using above formula (7),

$${M}_{priorart} = \frac{{B}_{h}}{{\sum }_{{k}_{m} - 1}^{K} [ {\sum }_{{k}_{p -} 1}^{{k}_{m}} ( {k}_{p} {λ}^{{k}_{m}} {e}^{- λ} [ ɛ N - {k}_{m} ] ! \frac{( \begin{matrix}r ɛ N \\ {k}_{p}\end{matrix} ) ( \begin{matrix}ɛ ( 1 - r ) N \\ {k}_{m} - {k}_{p}\end{matrix} )}{[ ɛ N ] !} ) ]} \qquad ( 9 )$$

The inventive process first isolates and pools all beads (hit and non-hit) from all microcompartments (incubation media or aliquots thereof), then sorts out hit beads. The number of necessary microcompartments from which the beads are isolated and pooled is assumed here as Mᵢₙᵥₑₙₜᵢₒₙ. The number of isolated and pooled beads (hit and non-hit) is λMᵢₙᵥₑₙₜᵢₒₙ, wherein λ is the abovementioned average bead population of the microcompartments, calculated according to above formula (2). Sorting then out from the pool the hit beads gives a number of hit beads which is rλMᵢₙᵥₑₙₜᵢₒₙ, wherein r is the abovementioned “library hit rate”. This number of hit beads is again designated as Bₕ for the purpose of comparing with the above prior art processes. Accordingly one obtains

$${M}_{invention} = \frac{{B}_{h}}{r λ} \qquad ( 10 )$$

From the comparison of (9) and (10) it follows that in order to obtain a desired number Bₕ of hit beads the inventive process requires a smaller number of microcompartments, Mᵢₙᵥₑₙₜᵢₒₙ, than the prior art processes require as their number of microcompartments, Mₚᵣᵢₒᵣₐᵣₜ, in those situations where:

$${\sum }_{{k}_{m} - 1}^{K} [ {\sum }_{{k}_{p -} 1}^{{k}_{m}} ( {k}_{p} {λ}^{{k}_{m} - 1} {e}^{- λ} [ ɛ N - {k}_{m} ] ! \frac{( \begin{matrix}r ɛ N \\ {k}_{p}\end{matrix} ) ( \begin{matrix}ɛ ( 1 - r ) N \\ {k}_{m} - {k}_{p}\end{matrix} )}{[ ɛ N ] !} ) ] < r$$

If the upper threshold value K is assumed as 1 then one obtains from the above inequality simply

e^{-λ}<1

which is independent of N, ε and r and is fulfilled for any λ>0. The λ must also be smaller than K, as already mentioned. For K=1 the useful range for λ is thus 0<λ<1, and in this case the inventive process outperforms the above prior art processes with respect to the required number of microcompartments in order to obtain a given number Bₕ of hit beads.

A preferred embodiment of the process of the invention is thus wherein:

1a) in step (1-a) the incubation medium has an upper threshold value K of 1 for the number of beads that can be contained therein, that is, the incubation medium is not allowed to contain more than one bead, or
1b) in step (i-b) each aliquot from the incubation medium has an upper threshold value K of 1 for the number of beads that can be contained therein, that is, each aliquot from the incubation medium is not allowed to contain more than one bead, and furthermore
2) the average bead population λ, defined as

$$λ = \frac{{{\sum }^{M}}_{i = 1} {( {k}_{m} )}_{i}}{M} \qquad ( 2 )$$

wherein
(kₘ)ᵢ is an integer number designating the number of beads in the i-th incubation medium or i-th aliquot of the incubation medium; M is the number of the incubation media or of the aliquots of the incubation medium, respectively; and the sum runs over all incubation media or over all aliquots of the incubation medium, respectively,
   is greater than 0 and smaller than 1.0.

The above requirement of K=1 is easily accomplished with rigid microcompartments such as microwells of appropriately small volume, as their rigidity enforces such K. For soft microcompartments such as droplets, the droplets with beads may be created right away with a sufficiently small volume such as to ensure K=1. Alternatively or in addition thereto the droplets may be further reduced in volume by a “droplet splitter” as used in ACS Comb. Sci. 21, pp. 425-435 (2019).

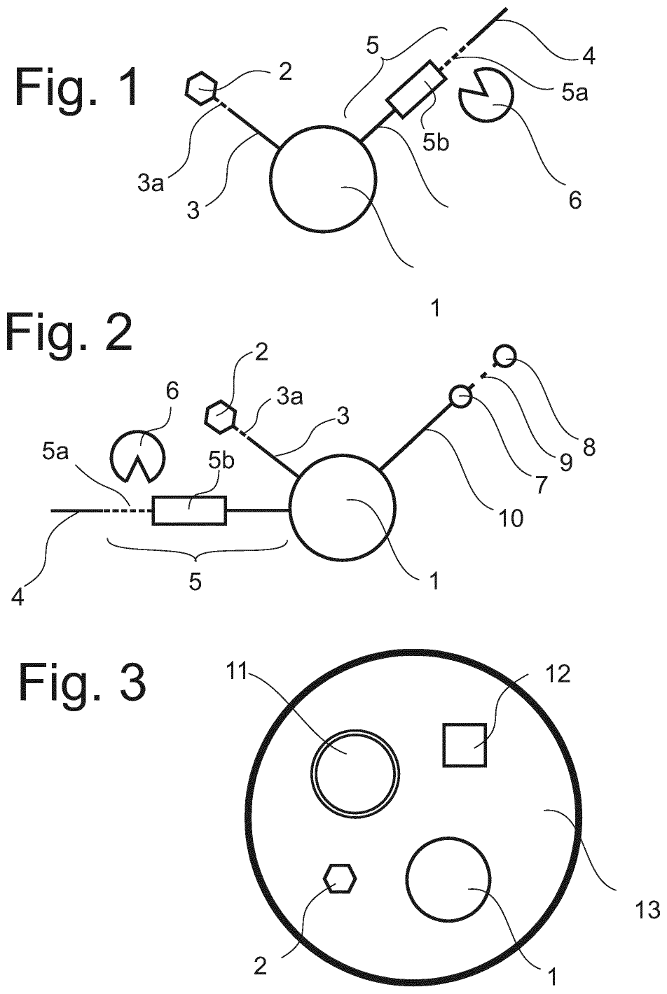

With reference to FIGS. 1, 2 and 3, herein a solution to the above problems are provided by associating to a bead 1 a small molecule in the form of a chemical structure 2, linked over a cleavable structure linker 3 to the bead 1. The structure linker 3 comprises a cleavable structure linker site (3a). The bead 1 furthermore comprises a DNA barcode 4 linked over a tag linker 5 to the bead 1. The tag linker 5 comprises a cleavable tag linker site 5a (FIG. 1) which is cleavable by a cleaving agent. In the screening process of the invention the cleavable tag linker site 5a is used, after incubation with the cellular target and after isolation and pooling of the beads, to cleave off all DNA barcodes 4 from the beads 1, which then allows their sequencing and correlation with corresponding chemical structures of the library. In the embodiment of FIG. 1 the tag linker 5 and/or the cleavable tag linker site 5a and or the encoding DNA 4 are assumed as being cleavable by the response molecule 12 generated during the incubation, and accordingly then the bead 1 is devoid of the (then unnecessary) probe. Alternatively, if neither the tag linker 5 nor the cleavable tag linker site 5a nor the encoding DNA 4 are cleavable by the response molecule 12, then the bead 1 furthermore comprises a chemical probe 7/8/9 linked over a spacer 10 to the bead 1, as shown in the embodiment of FIG. 2. Here, as shown in FIG. 2, the chemical probe 7/8/9 preferably consists of a fluorophore 7 and a quencher 8 for the fluorophore 7, wherein fluorophore 7 and quencher 8 are linked together over a response molecule-cleavable spacer 9. With reference to FIG. 3, the cellular target 11 is combined or co-encapsulated in an incubation medium 13 or in an aliquot thereof in a sample compartment together with the bead 1, and the chemical structure 2 is released from the bead 1 by cleaving the cleavable structure linker site (3a). Either the reaction of the chemical probe to the response molecule (FIG. 2) or the persistence of the tag linker in uncleaved form (both not shown in FIG. 3, but implicitly assumed as connected to the bead 1) records presence and/or activity of a response molecule 12 released (or released at a different level) by the cellular target 11 in the incubation medium 13 or aliquot thereof. FIG. 3 shows only one bead 1 in the compartment, which is a preferred embodiment of the invention, but there might also be several beads, each containing the same chemical structure. Analogously there might be several instances of the cellular target 11 instead of the sole one shown.

The screening process of the invention uses beads modified with the chemical structure of the library, the corresponding DNA barcode and optionally the probe for the response molecule. For purposes of the invention any type of particulate, solid or gel-like material could be used as the “beads”, provided the particulate material is

a) inert to the incubation medium in which the screening process of the invention is to be performed, in particular aqueous media (“inert” meaning the material does not react with the incubation medium and is essentially insoluble therein), and
b) has a particle surface containing chemically reactive moieties that allow covalent bonding of the chemical structure, the chemical probe and the DNA barcode over the respective linkers to the surface of the bead.

The bulk of the beads may be of inorganic particulate material (such as silica or alumina) in which case the the said chemically reactive moieties will primarily be hydroxyls. In another, more preferred embodiment the bulk of the beads are of an organic polymer, in particular of a polystyrene, which may have been modified on the surface by introduction of chemically reactive moieties. There are many such organic bead materials with modified surfaces available on the market, such as the beads marketed under the tradename Tentagel® by Rapp Polymere. These beads may typically contain a surface loading of chemically reactive moieties in the range of 0.1 to 0.5 mmol/g beads. The beads are preferably of approximately spherical shape, having preferably an average diameter in the range of 50 μm to 500 μm. These beads as such, even such ones with surfaces modified with chemically reactive moieties, are conventional.

The term “chemical structure” as used herein shall mean a small molecule in either free form or as a derivative thereof bound to a bead, whereby from the context in which the term is used it shall be clear which of the two meanings apply.

The screening process of the invention initially cleaves the chemical structures from the beads, so that the free, DNA-tagless chemical structures penetrate the cellular target. The chemical structure, once it has penetrated into the cellular target, may interact with, react with, interfere with, enhance or inhibit any system present in the cellular target. Such systems may e.g. be receptors of any kind, and systems involved in cell differentiation, transcription, translation, respiration, membrane construction, and mitosis.

The structure linker that connects the chemical structure to the bead can be any organic divalent radical bound on the one side to the bead surface and on the other side to the chemical structure. There is only the required feature that the structure linker contains a cleavable structure linker site, which allows to release the chemical structure from the bead in the incubation medium. It is preferred that said cleavable structure linker site is immediately adjacent to the chemical structure, such that upon cleavage the chemical structure contains as little as possible remaining residues from the structure linker.

Preferred examples of such cleavable structure linker sites are in the following table 1 (* indicates a valence preferably connected to the remainder of the structure linker and ** indicates a valence preferably directly linked to the chemical structure):

TABLE 1

| | | | residue left on the |
|---|---|---|---|
| | | | chemical structure |
| | | | after cleavage (if the |
| | | | chemical structure is |
| | | | immediately adjacent |
| | | | to the cleavable |
| Entry | cleavable structure linker sites | cleaving agent | linker) |
| 1 | *-S—S-** | Tris-(2- | HS— |
| | | carboxyethyl)- | |
| | | phosphine | |
| 2 | *-N═N-** | Na₂S₂O₄ | H₂N— |
| 3 | 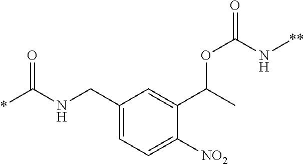 | UV 365 nm | |
| 4 | *-CH(OH)—CH(OH)-** | NaIO₄ | HC(O)— |
| 5 | 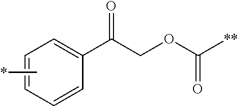 | UV 254 nm | HOOC— |
| 6 | 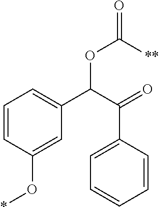 | UV 365 nm | |
| 7 | *-ENLYFQG-** (SEQ ID NO. 1) | TEV protease | H₂N-G- |
| 8 | *-5′-GTAACGATCCAGCTGTCACT-3′-** | Pvullendonuclease | 5′-CTGTCACT-3′- |
| | (SEQ ID NO. 2) | | |
| 9 | 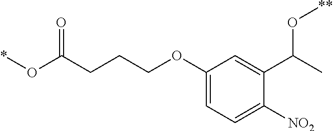 | UV 254 nm | HO- |

These cleavable structure linker sites as such are conventional and there are many more examples in the literature.

The bead used in the screening process of the invention firstly has the chemical structures of the library covalently bound to the bead surface over a cleavable structure linker. A chemical structure may be any chemical compound found in nature of generated by synthetic means or by (modified) biological activity like transcription. The molecular weight of the chemical structure may e.g. be in the range from a few hundred Dalton to several thousands or ten thousands of Daltons.

Preferred examples of chemical structures that could form part of a screening library are any pharmaceutically acceptable chemical compounds fulfilling all of the following a)-d):

a) they contain at the most five hydrogen atoms able to participate in hydrogen bonding and derived from hydroxy (providing one such hydrogen atom), primary amino (providing two such hydrogen atoms) and secondary amino (providing one such hydrogen atom);
b) they contain at the most ten oxygen and nitrogen atoms able to act as hydrogen bond acceptors (moieties containing an electron lone pair that is capable of participating in hydrogen bonding);
c) their molecular weight is at the most 500 Dalton; and
d) the negative decadic logarithm of its octanol/water partition coefficient (−log(c_{n-octanol}/c_{water}), wherein c_{n-octanol} is the molar concentration of the compound in n-octanol and c_{water} is its molar concentration in water, the n-octanol solution of the compound and the water solution of the compound being in contact with each other and in thermodynamic equilibrium at 25° C.) is at the most +5; preferably it is in the range of −0.4 to +5.
Compounds fulfilling the above conditions are usually defined as “rule of 5” compounds.

Preferably the screening process of the invention may rely on known libraries of chemical structures as candidates to be connected to the beads. A review of known libraries is given in Table 1 of J. Med. Chem. 59, pp. 6629-6644 (2016). For use as chemical structures in the instant process employing beads, the DNA tag connected over the amino group directly to the chemical structure, as shown in this table of this publication, will be replaced by a cleavable structure linker and bead connected thereto, as described herein. Furthermore, the split-and-pool methodology as described herein will be used to simultaneously construct the segments and building blocks of the chemical structures and the associated DNA tag segments.

The bead comprises a “plurality” of such chemical structures linked thereto. This plurality must be great enough such that the chemical structures, once released even from one sole bead, are sufficient in number, or are in high enough concentration in the incubation medium, such as to provoke a detectable release of response molecule from the cellular target. This “plurality” is limited by the number of reactive sites present on the surface of the bead. If it is not possible to obtain a detectable release of response molecules from the cellular target even when all reactive site of a bead are connected to chemical structures then that chemical structure may a priori be ineffective for its intended purpose and can be discarded from the assay. Furthermore the “plurality” of chemical structures may be in the range of 0.001 to 0.01 molar equivalent based on the above mentioned surface loading of the beads with chemically reactive moieties.

The bead used in the process of the invention secondly has the encoding DNA covalently linked over a tag linker to the bead surface. The tag linker may again be any suitable organic divalent linker, provided that it contains a cleavable tag linker site, which allows to release the DNA barcodes from the beads after their incubation with the cellular target, their isolation therefrom and their pooling. Examples for the cleavable tag linker site and their associated cleaving agent may either be as described above for the cleavable structure linker site and its associated cleaving agent, or, preferably it is a nucleotide sequence. More preferably the cleavable tag linker site is a nucleotide sequence which is cleavable by a restriction endonuclease as the cleaving agent, in which case that nucleotide sequence contains a recognition site for that restriction endonuclease. Many examples of suited restriction endonucleases and their associated recognition sites are known in the literature. E.g. table 2 of WO 2010/94036 A1 discloses in the second column assorted restriction endonucleases and their associated recognition and cleavage sites. A most preferred restriction endonuclease as the cleaving agent is Stu1.

In the case of a cleavable tag linker site which is cleavable by a restriction endonuclease it is furthermore preferred that the tag linker also comprises a PEG divalent spacer of 5 to 10 ethylene glycol units length, preferably of 8 ethylene glycol units, immediately adjacent to the cleavable tag linker site. This may favour the cleavage of the recognition site by the restriction endonuclease.

It is preferred that said cleavable tag linker site is immediately adjacent to the encoding DNA, such that upon cleavage the encoding DNA contains as little as possible remaining residues from the tag linker. Preferably the tag linker itself is constructed over the so-called “click chemistry” reactions.

The above preferred variants of tag linker, cleavable tag linker site and DNA barcode, optionally with said PEG spacer forming part of the tag linker and being immediately adjacent to the cleavable tag linker site is preferably constructed according to the following synthetic scheme:

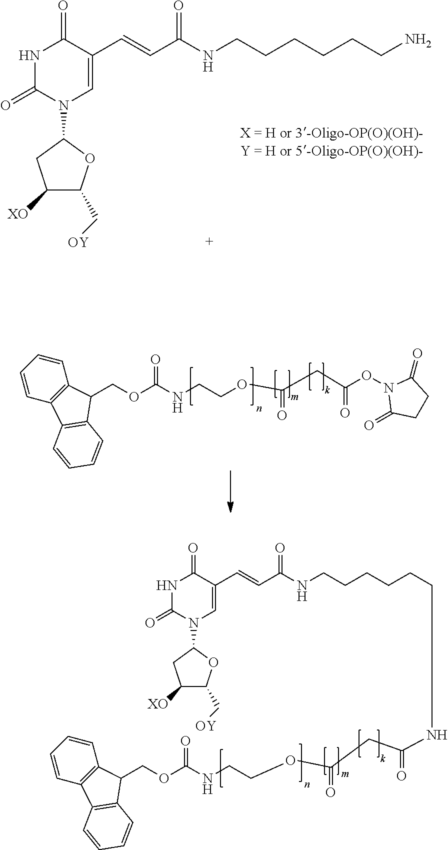

In this scheme the Fmoc-PEG derivatives are commercially available, e.g. from JenKem (USA), Abbexa (GB) or Iris Biotech (DE). Therein, n is from 5 to 10 and preferably is 8. The m is 0 or 1 and the k is 0 to 2; preferably both m and k are 0, or m is 0 and k is greater than 0. The C₆-amino-terminated deoxythymidine (dT) derivatives are also commercially available, e.g. from GeneLink (USA). The Fmoc-protected amine moiety may thereafter be deprotected and the free amine be connected to a DNA tag linker already connected to the bead, or alternatively the free amine may directly be connected to beads having carboxyl surface functionalites, in which case the PEG linker itself will form the DNA tag linker. The one or two oligo's as X and/or Y may serve as headpiece DNA's onto which in the split-and-pool methodolog described herein further encoding DNA segments may successively be connected. If both X and Y are DNA oligos, then this may be subsequently used to build up a DNA tag in the form of a hairpin DNA. The cleavable tag linker site may e.g. be formed by converting the double bond near the deoxythymidine moiety into a vicinal diol using alkaline hydrogen peroxide, which will then be a cleavable tag linker site cleavable by NalO₄ (see Table 1 above). Alternatively, the oligo's as X and/or Y may contain a restriction endonuclease recognition site as the cleavable tag linker site, as discussed above.

For both the processes of the invention and for the bead of the invention the cleavable structure linker site and the cleavable tag linker site are “orthogonal” in reactivity, that is, the cleavable structure linker site is not cleavable under reaction conditions that cleave the cleavable tag linker site, and vice versa;

The tag linker and/or the cleavable tag linker site and/or the encoding DNA may be cleavable by the response molecule produced during the incubation, in which case the chemical probe is unnecessary. The cleavable tag linker site itself is then mandatorily cleavable firstly by the cleaving agent and furthermore optionally by the response molecule. Here, the cleaving agent and the response molecule may be different or be the same; preferably they are different.

The encoding DNA itself is preferably a hairpin DNA. This allows construction of a fully double-stranded DNA from only one DNA strand, because it pairs itself to the double strand. The construction of only one encoding DNA sequence is thus necessary. The encoding DNA may comprise, besides the actual encoding sequences characteristic for a chemical structure to be encoded, further leading and/or trailing sequences that may be necessary or beneficial in connecting the encoding DNA to the bead and/or in cleaving the tag linker at the cleavable tag linker site. These further leading and/or trailing sequences will typically be identical for all encoding DNA instances.

The bead comprises a “plurality” of such encoding DNA instances. This plurality must be great enough such that the encoding DNA instances even from one sole bead are sufficient in number such as to allow amplification, e.g. by PCR, and subsequent sequencing. Furthermore the “plurality” of encoding DNA instances may be in the range of 0.001 to 0.01 molar equivalent based on the above mentioned surface loading of the beads with chemically reactive moieties.

The bead used in the invention optionally comprises a chemical probe that is susceptible to the response molecule and which is linked to the bead. The chemical probe can be a substrate of the released response molecule, which can be an enzyme, protein or molecule, such that the beads fluoresce (or cease to fluoresce) after incubation with the released material and appropriate additives. Alternatively (or in conjugation with), the released enzyme may degrade of alter the DNA barcode conjugated to the bead in such a manner that its detection via amplification or hybridization is modulated. The probe may comprise chemical indicators, including reporter molecules. It may be, for example, colorimetric (i.e. result in a coloured reaction product between response molecule and probe that absorbs light in the visible range), fluorescent (e.g. based on an enzyme that converts the response molecule and probe to a reaction product that fluoresces when excited by light of a particular wavelength) and/or luminescent (e.g. based on bioluminescence, chemiluminescence and/or photoluminescence). Preferably the probe reacts by fluorescence upon contact with the response molecule and is thus either a fluorophore or a combination of a fluorophore with an associated quencher, wherein the response molecule cleaves the quencher from the fluorophore. However the chemical probe is only present if the bead does not comprise a tag linker which by itself is cleavable by the response molecule.

The spacer that connects the probe to the bead may again be as described above for the structure linker and/or tag linker.

The cellular target is preferably a prokaryotic or eukaryotic cell. More preferred examples of prokaryotic cells are Gram-positive bacteria, Gram positive Cocci, Gram negative Cocci and Gram negative bacteria. More preferred example of eukaryotic cells are fungal cells and animal cells, in particular human cells. The cellular target contains proteins, receptors, or other species that are liable to interact with a chemical structure that penetrates from the outside of the cellular target into the cellular medium, following which interaction the cellular target may start to produce a response molecule, may increase the production of a response molecule, or may lower or even discontinue the production of a response molecule.

The bead used in the screening process of the invention is synthesized according to known chemistries either by directly bonding the encoding DNA over the tag linker and the chemical structure to the linker to the bead.

Alternatively, the encoding DNA and the chemical structure may be synthesized step-wise directly on the beads, e.g. by split-and-pool synthesis. In this alternative initially a “scaffold” (a chemical core structure common to all small molecules of the library to be tested) is linked to the bead over a cleavable linker as described above and the tag linker, yet devoid of any encoding DNA, is also attached to the bead. The scaffold is typically a ring-containing structure which contains a set of diversity sites, such as typically 1 to 3 diversity sites. Each such diversity site is a functional group onto which a variety of different substituents can be connected over one and the same synthetic route. The reactivity of the diversity sites, if there is more than one, is “orthogonal” that is, one diversity site may be reacted under its specific reaction conditions with said variety of substituents without affecting the other diversity sites, which are reactive only under reaction conditions different of those for the said one diversity site. The bead batch containing the scaffold so introduced is split into as many sub-batches as there are first substituents in the chemical library, and each sub-batch is modified with one such first substituent, and simultaneously or following that an associated first encoding DNA increment is added to the tag linker. The sub-batches are then re-pooled to one single batch. Re-splitting into sub-batches equal in number to a further substituent of the library, connecting the further substituent and a further associated encoding DNA increment to the DNA tag already bound to the beads, employing a customary ligase such as T4 ligase, and re-pooling is repeated for as many times as there are further substituents to be connected to the scaffold, that is, until all diversity sites in the scaffold have been modified by respective substituents of the chemical library.

Examples of diversity sites for the scaffold are according to the following table 2 (* indicates possible connection point(s) of the diversity site to the scaffold):

TABLE 2

| No. | diversity site | variable substituent | product | Method |
|---|---|---|---|---|
| 1 | 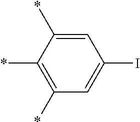 [SMILES: *c1cc(I)cc(*)c1*] | H—≡—R^{x} | 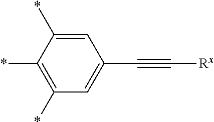 | Sonogashira |
| 2 | | 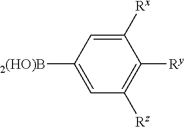 | 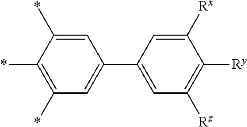 | Suzuki |
| 3 | *-N₃ | R^{x}—≡—R^{y} | 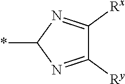 | Huisgen |
| 4 | | OHC—R^{x} | *-NH(CH₂R^{x}) | Reductive |
| | | | or | amination |
| | | | *-N(CH₂R^{x})₂ | |
| 5 | *-NH₂ | 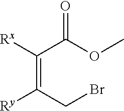 | 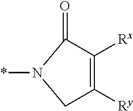 | 20 eq. bromide, 60° C., 2 h |
| 6 | 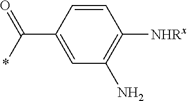 | OHC—R^{y} | 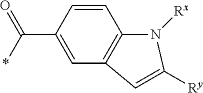 | 60 eq. aldehyde, 60° C., 18 h |
| 7 | *-NH—C(O)—C(R^{x})—NH₂ | | 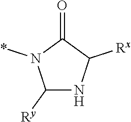 | |
| 8 | *-(NH)_{q}—C(S)—NH₂ (q = 0 or 1) | 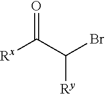 | 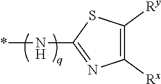 | 50 eq. bromo- ketone, rt, 24 h |

The above diversity sites and reactions are known to be compatible with DNA moieties present in the same molecule or reaction medium and can thus be modified in the presence of the encoding DNA (see Satz, A. L., Cai, J., Chen, Y., Goodnow, R., Gruber, F., Kowalczyk, A., Petersen, A., Naderi-Oboodi, G., Orzechowsky, L., Strebel, Q., in Bioconjugate Chemistry 26, pp. 1623 ff. (2015)).

In the process of the invention the incubation of beads and cellular target in an incubation medium may be accomplished by either: Variant a): providing an incubation medium and combining therein one more beads containing multiple instances of one sole defined chemical structure and its associated encoding DNA and the cellular target, and running the incubation in that sole incubation medium (this alternative requires one incubation medium for each chemical structure and each type of cellular target). As “incubation medium” is understood here a medium volume which as such is sufficiently small to be contained in a microcompartment, such as a microwell or a droplet dispersed in oil or microspotted or ink-jetted, but which is sufficiently large to contain at least one bead.

Variant b): providing an incubation medium and combining therein all beads containing multiple instances of all chemical structures of the library and the cellular target, and dividing off from that incubation medium aliquots (this alternative requires one incubation medium for all chemical structures and each type of cellular target). Here, as “aliquot of an incubation medium” is understood a small volume portion divided off from an incubation medium of macroscopic size and not necessarily representing a defined volume fraction of the macroscopic incubation medium, which is sufficiently small to be contained in a microcompartment, such as a microwell or a droplet dispersed in oil or microspotted or ink-jetted, but which is sufficiently large to contain at least one bead.

Having two or more beads in one aliquot might give after incubation thereof an ambiguous result in the prior art assays, if the beads are of different types. This is however not detrimental for the process of the invention, because the inventive process first isolates all beads (hits and non-hits) anyway and only then sepates hits from non-hits. There will always be many other aliquots each of these containing one individual bead of the said different types. Typically there will be several tens of aliquots that contain a bead of a given type, whether alone (the predominant situation) or which contain that bead in combination with one or more beads of another type. The aliquots may be present in sample compartments which may take any form including a well of a microplate, microfabricated nanowells, an aqueous droplet in oil, or compartments comprised of lipid bilayers. These sample compartments as such are conventional. The aliquots may be prepared by co-spotting of cellular targets and beads in the incubation medium via mechanical microspotting in which case the sample compartment may simply be defined as aliquot droplets separated from each other by a spatial distance. Alternatively, aliquots may be generated from a bulk incubation medium containing the cellular target, the beads and optional additives in the form of aqueous droplets as a water-in-oil emulsion, wherein each monodisperse droplet contains preferably only one bead.

Only one bead is sufficient for carrying out the assay of the invention in each incubation medium or aliquot thereof. Accordingly an average bead population λ of about 1.0, preferably about 0.2 to about 0.9, is a preferred embodiment, wherein λ is defined and calculated as described in the introduction.

The cellular target may be preferably added to the incubation medium before dividing it up into said aliquots. If the cleaving conditions for cleaving the chemical structure from the bead(s) are detrimental to the cellular target then it might on the other hand be preferable to add the cellular target to the already divided aliquots, after having cleaved off the chemical structures from the bead(s) contained therein and optionally even after having inactivated any remains of harmful cleaving chemicals.

The cleaving of the chemical structure from the bead(s) must however be carried out after said dividing into aliquots, to maintain a confinement of cleaved chemical structure and bead-bound encoding DNA tag.

The incubation medium itself will normally be an aqueous medium, typically containing further adjuvants and/or nutrients (including enzyme and potential modulators for the cleaved off chemical structure), needed to carry out the assay and/or the maintain the viability of the cellular target. The incubation is typically carried out under conditions that allow to maintain the viability of the cellular target, such as or near at room temperature and physiological pH and salt concentrations, for a time until a possible release or change of release of response molecule is observed in the incubation medium or in one or more of the aliquots thereof, whether by reaction of the probe attached to the bead, by appearance of cleaved DNA in the incubation medium, or any other technique suited to detect the presence or amount of response molecule in the incubation medium (such as GC-MS). In the process of the invention it is preferred, or even essential, that that any reaction of the response molecule with the probe, or, as the case may be, any cleavage of the tag linker and/or the cleavable tag linker site and/or the encoding DNA by the response molecule, is kinetically irreversible. Namely, in the case of a reversible reaction, the reacted probes would convert back to unreacted probes; or in the case of a cleaved tag linker and/or cleaved tag linker site and/or cleaved encoding DNA these would be reconnected by simple reestablishment of the thermodynamic equilibrium, once the response molecule was separated from the beads, as outline above.

Following the incubation there are two alternatives for the pooling of the beads, isolation of hit beads, characterisation of the encoding DNA and correlation with chemical structures of the library:

Alternative a (if the tag linkers and/or the cleavable tag linker sites and/or the DNA sequences are cleavable by the response molecule): Once any released encoding DNA or fragments thereof are detected in the incubation medium or in any aliquot thereof, or in a significant number of aliquots, the beads are isolated from all incubation media or are isolated from all aliquots thereof, and then pooled. This pooling eliminates the dependency of Pₕ discussed in the introduction from the average bead population λ that was present in the incubation media or aliquots thereof. This isolation also serves to separate the beads from any remains of response molecule, and such isolation is not possible employing processes described in the prior art. Isolation of the beads from the aliquots quenches the assay at the desired time point, and then allows for later analysis of the beads in a pooled state (i.e. PCR amplification and sequencing). Such follow-up analysis is inherently impossible employing processes described in the prior art. In the absence of such separation the response molecules would continue to interact with the pooled beads and produce further cleavage of DNA linkers attached to all pooled beads, which would make the identification of the beads which had the active chemical structure attached thereto impossible. In this alternative a) the DNA of all isolated beads is released from the beads by cleaving the cleavable tag linker sites. Following that, all released DNA material is analysed and sequenced. There will mostly be full-length instances of released encoding DNA (if during incubation the response molecule did not cleave neither the tag linker nor the cleavable tag linker site nor the encoding DNA). These full-length instances of released encoding DNA have thus been released from beads that carried a chemical structure that was inactive during incubation. Furthermore there will be released fragments of encoding DNA (if during incubation the response molecule cleaved the encoding DNA itself) and encoding DNA sequences that are totally absent in the released DNA material (if during incubation the response molecule cleaved the tag linker or the cleavable tag linker site). These released fragments of encoding DNA and any full-length instances encoding DNA that are completely absent in the released DNA material are thus from beads that that carried a chemical structure that was active during incubation. The correlation to active chemical structures in the library is thus made from fragments of encoding DNA present in the released DNA material and from full-length instances of encoding DNA that are completely absent in the released DNA material.

The extent of removal of encoding DNA during incubation of a screening batch of beads may be based on comparison with respect to a control batch of beads. The control batch is simply DNA-sequenced without any incubation, or the control batch is optionally incubated before DNA sequencing under the same conditions as the screening batch, except that the cellular target is absent. Both the screen and the control are carried out using the same overall number of beads. More preferably here, the number of beads in the screening batch and in the control batch is 20-100 times the number of chemical structures in the assayed chemical library, thus c defined in the introduction is in the range of 20 to 100. If here at least 5 beads carrying one given chemical structure are found in the control batch (that is, the full corresponding DNA tag could be found in these at least 5 beads) and less than 2 beads carrying that chemical structure are found in the screening batch (that is, the full corresponding DNA tag could be found only in these less than 2 beads), then that chemical structure may be considered a possible hit. If essentially all beads carrying one given chemical structure are found in the control batch (that is, the full corresponding DNA tag could be found in a number of beads corresponding essentially to the number of beads employed for each chemical structure) and essentially no beads carrying that chemical structure are found in the screening batch (that is, the full corresponding DNA tag could be found in essentially none of the beads of the screening batch), then that chemical structure may be considered a clear hit.

Alternative b (if the beads have a chemical probe susceptible to the response molecule covalently linked thereto): Once the incubation in the incubation medium or in the aliquots thereof has proceeded sufficiently, as described above, all beads are isolated from all incubation medium or aliquots thereof and so separated from any remains of response molecule, and then pooled. This pooling eliminates the dependency of Pₕ from the average bead population λ that was present in the incubation media or aliquots thereof. This isolation also serves to separate the beads from any remains of response molecule In the absence of such separation the response molecules would continue to interact with the probes of further pooled beads and produce further responses, which would make the identification of the beads which had the active chemical structure attached thereto impossible. Additionally, such isolation is not possible employing processes described in the prior art. Isolation of the beads from the aliquots quenches the assay at the desired time point, and then allows sorting of the collected beads to be optimized and accomplished at a later time of choosing. In this alternative b) the beads having reacted probes are then isolated from beads having unreacted probes. If the reacted probes have fluorescence and the non-reacted probes do not have fluorescence (or vice versa) then such sorting out may be done using a commercially available fluorescence-activated cell sorter (100 million beads may be sorted in just hours, a rate 100-fold greater than described in the prior art). The use of a commercially available fluorescence-activated cell sorter is not possible when employing processes described in the prior art, and its higher throughput is required to interrogate numerically large chemical libraries. In this alternative b) the DNA of all isolated beads having reacted probes is released from the beads by cleaving the cleavable tag linker sites. Following that, all released DNA material is analysed and sequenced. There will only be full-length instances of released encoding DNA. The correlation to active chemical structures in the library is thus made from all full-length instances of encoding DNA that are present in the released DNA material.

In alternative b) the number of beads employed for each chemical structure of the library is preferably in the range of 5-10, thus c defined in the introduction is in the range of 5 to 10

In either of the above isolation alternatives a) or b) the incubation reaction may be terminated, if desired, before or as the first step in isolation of the beads, e.g. by destroying or inactivating of auxiliary agents and/or the response molecule. Such destruction or inactivation may e.g. be done by denaturing agents, such as ethanol, by heat, by evaporation, precipitation, change in pH, oxidative destruction or salting out.

In either of the above isolation alternatives a) or b) the pooling is either from all used incubation media (above described incubation variant a) or from all used aliquots thereof (above describe incubation variant b).

In either of the above isolation alternatives a) or b) any released encoding DNA or released fragment thereof is preferably amplified by standard techniques such as PCR, and sequenced using standard protocols. Next generation high-throughput sequencing may provide 200 million sequence reads per sequencing lane. In this manner, a ten-fold increase in the number of samples investigated will have little effect on processing times, and so ten million small-molecules can be investigated as readily as one million. Thus, the process of the instant invention is scalable with respect to both logistics and processing times.

The screening process of the invention may e.g. serve to screen for:

a) Lethality of the chemical structures of a compound library against a pathogenic cellular target. In this case the response molecule may be a protein indicative of death or apoptosis of the cellular target, or it may be a degradation product from the cellular target itself. Onset of release of the response molecule during incubation of the cellular target with the is indicative of lethality.

b) Beneficiality of the chemical structures of a compound library in a cellular target. In this case the response molecule may be any compound known to be released at a certain level by the healthy cellular target, and known to be released at a higher (respectively lower) level in a compromised cellular target. Lowered (respectively increased) release of the response molecule during incubation of the cellular target with the is indicative of beneficiality.

c) Suitability of a chemical structure as a prodrug that is able to penetrate the cellular target, in case where the drug itself is incapable of penetrating the cellular target.

A first preferred application for the process of the invention is the secreted embryonal alkaline phosphatase (SEAP) reporter assay. This assay uses genetically engineered reporter cells that indirectly report an internal activation by a chemical structure over an enhanced expression of SEAP which is then secreted from the cellular target into the incubation media or aliquots thereof. Such genetically engineered reporter cells can on the one hand be custom designed according to known technologies. Published examples are e.g. the Huh7.5-EG(_4B5A)SEAP cell line described by Pan K L., Lee J C., Sung H W., Chang, T Y., Hsu, J T A; Antimicrob. Agents. Chemother. 53(11) pp. 4825-4834 (2009) (secretes SEAP upon infection by hepatitis C viruses) and the 293E/CRE-SEAP cell line described by Durocher D., Perret S., Thibaudeau E., Gaumond M H., Kamen A., Stocco R., Abramovitz M., Anal. Biochem. 284, pp. 316-326 (2000) (secretes SEAP upon activation of G-protein-coupled receptors, an important target for many known drugs). Such engineered cells are in some cases even available on the market. Examples therefore are the cell lines HEK-Bue™ IFN-α/β (expresses and secretes SEAP upon stimulation with human IFN-α or IFN-β) designed are the THP1-Blue™ ISG cell lines marketed by InvivoGen (express and secretes SEAP upon activation of stimulator of interferon genes (STING), whose activation is important in treatment of cancer and infectious diseases). All these cell lines can be identically used also in the inventive SEAP assay.

The chemical structure to be used as candidates in an inventive SEAP assay and to be linked to the beads can on the one hand compounds of the above mentioned “rule of 5” type. As second preferred category are any compounds that can be synthesized using known DNA-compatible chemistries. A third preferred category of chemical structure candidates are macrocyclic peptides. A library of cyclic peptides has been disclosed e.g. in ACS Chem. Biol. 13, pp. 53-59 (2017). For use as chemical structures in the instead process and bead, the DNA tag connected over the amide group directly to the cyclic peptide, as shown in this publication, will be replaced by a cleavable structure linker and bead connected thereto, as described herein. A further exemplary subset of chemical structure candidates may e.g be according to one of the following libraries indicated the following Table 3

TABLE 3

| Scaffold (** indicates the connection point | |
|---|---|
| to the cleavable structure linker) | |
| 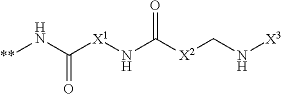 | Examples for diversity site X³ are hydrogen, natural or unnatural amino acids (preferably linked to the scaffold amino over their carboxyl group); and di- or tripeptides made up of natural amino acids (preferably linked to the scaffold amino over their C-terminus). |
| 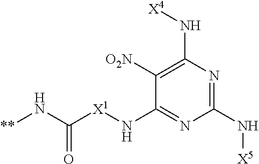 | Examples for diversity sites X⁴ and X⁵ are, independently from each other, hydrogen, acetyl, formyl, and natural or unnatural amino acids (preferably linked to the aromatic amino over their carboxyl group); and di- or tripeptides made up of natural amino acids (preferably linked to the aromatic amino over their C-terminus)). |
| 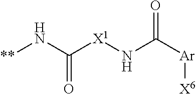 | Examples for diversity site X⁶ are hydrogen, hydroxy, amino, C₁-C₄-alkylcarbonyl, nitro, C₁-C₄-alkylester, C₁-C₄-alkoxy, formyl, and natural or unnatural amino acids Examples for diversity site Ar are phenyl, pyridyl, thienyl, furanyl, pyridiminyl, imidazolyl, pyrrolyl and their respective benzo-, pyridinyl- and pyrimidinyl-fused derivatives, whereby the fusing phenyl, pyridinly or pyrimidinyl may itself be unsubstituted or substituted by 1-3 substituents as exemplifed for X⁶ |
| 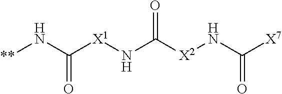 | Examples for diversity site X⁷ are hydrogen, hydroxy, amino, C₁-C₄-alkyl, C₁-C₄- alkoxy, formyl, natural or unnatural amino acids (preferably linked to the scaffold carbonyl over their amino group); and di- or tripeptides made up of natural amino acids (preferably linked to the scaffold carbonyl over their N-terminus). |

Examples for the diversity sites X¹ and X² appearing in all of them may be, independently from each other, e.g. —O—, —NH—, —(CH₂)—, natural or unnatural amino acids (preferably linked to the respective scaffold carbonyl over their amino group and linked to the respective scaffold amino over their carboxyl group); and di- or tripeptides made up of natural amino acids (preferably linked to the respective scaffold carbonyl over their N-terminus and linked to the scaffold amino over their C-terminus).

The linking of these chemical structures over a cleavable structure linker may be done analogously as outlined above under Table 1.

The probe to be linked to the beads in the inventive SEAP assay may on the one hand be an organic, in particular aromatic or polyaromatic, hydroxyl-containing fluorophore or chromophore, wherein the hydroxyl(s) have been converted to phosphates being susceptible to cleavage by the released SEAP. Upon cleavage by SEAP released into the incubation media or aliquots the phosphate groups are hydrolysed, thus enabling fluorescence of the fluorophore or a coloration or color change of the chromophore. Known examples of such phosphate-quenched fluorophores are 1-oxo-3′,6′-diphosphonooxy-spiro[isobenzofuran-3,9′-xanthene]-5-carboxylic acid and 1-oxo-3′,6′-bis(phosphonooxymethoxy)spiro[isobenzofuran-3,9′-xanthene]-5-carboxylic acid). These phosphorylated chromophores or fluorophores preferably also either contain a carboxylic acid group that enables linking to the beads by amide group formation using a suited amino-group terminated linker which has already been connected to the beads. Incumbent fluorescence is easily detectable in the pooled beads as in the corresponding homogeneous prior art assay. Incumbent coloration, or incumbent colour change, of bead-bound chromophores may be measured on the pooled beads e.g. by reflectance spectroscopy instead of normal spectroscopy as would be used in the corresponding homogeneous prior art assay.

A second preferred application for the screening process of the invention is in screening for antibiotics. Here the cellular target will be a pathologic bacterium that releases upon cell death a nuclease as the response molecule. In this first application the bead may preferably comprise multiple instances of one sole chemical structure and multiple instances of encoding DNA sequences being covalently linked to the bead over tag linker containing a moiety which is cleavable by that nuclease. Apart from that, the bead is devoid of any other chemical moiety susceptible, cleavable and/or reactive to that response molecule. That is, the bead may consist of a bead core, the linked chemical structures and the linked encoding DNA but is devoid of anything else. Such bead is itself an object of the invention. Here either a plurality of possible antibiotic candidates may be linked in one step to an equal number of beads (or an equal number of bead batches) and simultaneously the corresponding encoding DNA tags may be connected to each such bead (or the beads in each bead batch). Alternatively the beads may be constructed stepwise, using a suited scaffold which has a high probability of producing an antibiotically active chemical structure upon being modified with further substituents. Here such scaffold may e.g. be a cyclic oligopeptide containing e.g. lysine, asparagine and/or serine units that provide e.g. hydroxy and/or amino as diversity sites for attaching further substituents.

A third preferred application for the screening process of the invention is in screening of small molecules for possible anticancer drugs. An effective anticancer drug may induce apoptosis of targeted cancer cells. Such apoptosis is accompanied by release of caspases. In this second particularly preferred application, the bead of the invention may preferably comprise as the probe, in analogy to the microparticle-based system described by Yozwiak C. E, Hirschhorn, T. and Stockwell, B. R. in ACS Chem. Biol. 2018, 13, 761-771, a moiety of the following formula:

447

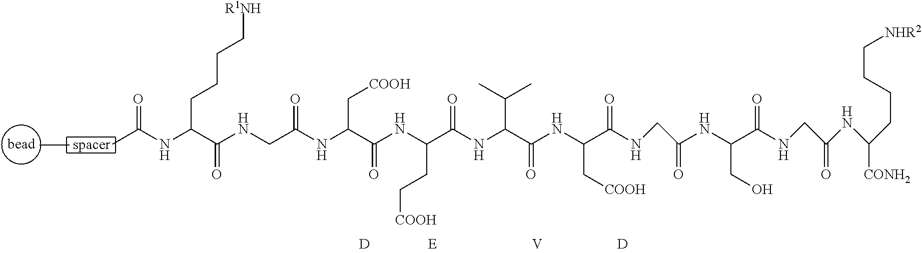

wherein “spacer” is a divalent residue; R¹ is a fluorophore, and R² is a quencher acting by fluorescence resonance energy transfer (FRET) or by contact quenching onto the fluorophore as R¹. The peptide sequence therein is SEQ ID NO. 3. This probe is cleaved in case of release of caspase-3 at the rightmost D amino acid of the DEVD sequence, which removes the quencher as R² and enables fluorescence by R¹. Fluorescent beads may be sorted out using a commercial fluorescence activated cell sorter (FACS). The positively sorted beads are subjected to DNA-amplification and sequencing, to find out to which small molecule was initially connected to these beads and caused apoptosis and caspase release. Preferably, in the above formula R¹ and R² are selected according to one of the rows of the following table:

| R¹ | R² |
|---|---|
| 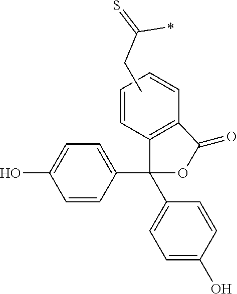 | 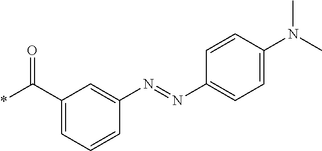 [SMILES: *C(=O)c1cccc(/N=N/c2ccc(N(C)C)cc2)c1] |
| 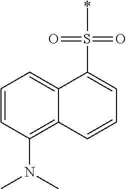 [SMILES: *S(=O)(=O)c1cccc2c(N(C)C)cccc12] | 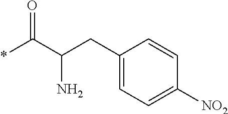 [SMILES: *C(=O)C(N)Cc1ccc([N+](=O)[O-])cc1] |

The invention will now be illustrated by the following non-limiting examples.

### Example 1: Assay for Fluoroquinolones Activity Against Bacteria

The test is performed with fluoroquinolones that might induce oxidative stress on bacteria, such as ciprofloxacin or levofloxacin. Oxidative stress may cause DNA damage and thus release of endonucleases, such as BapE DNA endonuclease, from the oxidatively stressed bacteria.

Step 1: Preparation of Barcode DNA's and Attachment Thereto to Beads (Protocol for Each Individual Barcode DNA, Corresponding to One Fluoroquinolone to be Assayed)

A respective headpiece DNA, each containing a unique nucleotide sequence to “tag” one respective fluoroquinolone is provided, and is functionalized with a 8 PEG chain and a primary amine as described in the general part of the application. To a solution of this functionalized headpiece DNA (1 mM in H₂O) is added, at 0° C., Hünigs' base (5 equiv.) and (1R,8S,9S)-Bicyclo[6.1.0]non-4-yn-9-ylmethyl N-succinimidyl carbonate (3 equiv.) delivered as a 0.2 M solution in NMP. Upon reaction completion, as judged by ESI-TOF mass spectrometry, the reaction is purified following reported precipitation procedure. The resulting functionalized DNA headpiece (in the following DNA-BCN), is re-suspended in water at a concentration of 1 mM and used without further purification.

Tentagel beads functionalized with azidopentanoic acid (loaded at 0.29 mmol/g), are split into a number of batches corresponding to the number of fluoroquinolones to be assayed. The beads within each batch are washed briefly with PBS, then water and finally CH₃CN. Each of the DNA-BCN's from the preceding step (0.004 equiv.) is dissolved in a 1:1 mixture of PBS:CH₃CN (alternatively 10% Pyridine in water can be used), and added to one of the batches with the azide-modified beads. Strain promoted alkyne azide cycloaddition (SPAAC) is then carried out at 45° C. in an incubator for about 24 hours. The DNA-BCN-functionalized bead batches are washed with CH₃CN, PBS and finally with molecular biology-grade water and kept at 0° C. in an ice bath until further use.

Step 2: Attachment of Fluoroquinolones to Beads

All DNA-BCN-functionalized bead batches obtained from step 1 are subject to reduction of remaining azido moieties to primary amine moieties using triphenyl phosphine in DCM and H₂O at room temperature.

Each bead batch so obtained is reacted with one respective fluoroquinolone antibiotic according to the following protocol. The beads are subjected to standard peptidic coupling and functionalized with an acid-labile Rink linker followed by a PEG8 linker and two lysine residues. The beads of each batch (1.0 equiv., 0.0096 mmol) are treated with a pre-activated solution of 4-(4-(1-hydroxyethyl)-2-methoxy-5-nitrophenoxy)butanoic acid (a secondary alcohol photolabile linker; 4.0 equiv., 38.4 μmol), HATU (3.8 equiv., 36.5 μmol) and Hünig's base (6.0 equiv., 57.6 μmol) in 100 μl of NMP for 2-12 hours at room temperature. The beads are then washed carefully and an analytical sample is checked by LCMS upon acid-mediated cleavage. A solution of the fluoroquinolone antibiotic (10.0 equiv., 48.0 μmol), DIAD (15.0 equiv., 72.0 μmol) triphenylphosphine (15.0 equiv., 72.0 μmol) in 150 μL of anhydrous THF and a small amount of NMP to help dissolve the fluoroquinolone is prepared and the Mitsunobu complex formation is run at 0° C. The solution so prepared is poured over the beads (1.0 equiv., 0.0048 mmol). Reaction is run initially at 0° C. and then at room temperature for 12 hours, to substitute the hydroxyl of the already bead-bound photolabile linker by the carboxylate from the fluoroquinolone. The beads are then washed carefully and an analytical sample is checked by LCMS upon acid-mediated cleavage. The beads of all batches are finally pooled, to obtain one single pool containing beads each being modified with one single unique fluoroquinolone/DNA-BCN combination.

Step 3: Concapsulation of Beads and Bacterial Cells and Assay (Protocol for Each Bacterial Strain to be Tested)

Using a flow focusing microfluidic chip, aqueous droplets are generated in oil to yield an emulsion of monodisperse droplets. The aqueous phase contains cells of the bacterial strain in question, fully functionalized and barcoded beads as prepared above, and additives, such that each droplet contains 0, 1, or several of the beads and 0, 1, several, or up to ten thousands of bacterial cells. Droplets are collected in a small reaction tube. Droplets with beads are exposed to several minutes of UV-light to liberate the fluoroquinolones from all the beads by photolytic cleavage of the above photolabile linker. The droplets are incubated several hours. The droplets are then optionally (depending on the desired assay reaction) heat inactivated or the emulsion is broken by organic solvent, which also stops the detection reaction. Beads are collected on a frit and the filtrate is amplified by PCR and sequenced to test for the presence of any headpiece DNA's that possibly were cleaved by bacterial endonuclease, indicating that the fluoroquinolone associated to that headpiece DNA was active against that bacterium.

### Example 2: Sequential Construction of Bead-Linked Encoding DNA in View of Split-and-Pool Synthesis of Beads Containing Linked Library of Chemical Structures and Linked Encoding DNA

To a solution of a headpiece DNA oligomer functionalized with a 8 PEG chain and a primary amine (1 mM in H₂O) and a primary amine as described in the general part of the application are added, at 0° C., Hünigs' base (5 equiv.) and (1R,8S,9S)-Bicyclo[6.1.0]non-4-yn-9-ylmethyl N-succinimidyl carbonate (3 equiv.) delivered as a 0.2 M solution in NMP. Upon reaction completion, as judged by ESI-TOF mass spectrometry, the reaction is purified following reported precipitation procedure. The resulting functionalized DNA headpiece (DNA-BCN), is re-suspended in water at a concentration of 1 mM and used without further purification.

Tentagel beads functionalized with azidopentanoic acid (loaded at 0.29 mmol/g), are washed briefly with PBS, then water and finally CH₃CN. The DNA-BCN from the previous step (0.004 equiv.) is dissolved in a 1:1 mixture of PBS:CH₃CN (alternatively 10% pyridine in water can be used), and added to the Tentagel beads. Strain promoted alkyne azide cycloaddition (SPAAC) is then carried out at 45° C. in an incubator for about 24 hours, to give beads having an initial headpiece DNA oligomer connected thereto.

Beads resulting from the preceding step are washed with CH₃CN, PBS and finally with molecular biology-grade water. While preparing a ligation mixture, the beads are kept at 0° C. in an ice bath. The further tagging DNA to be ligated to the existing DNA tag (1.2 equiv. with respect to the DNA loading from the preceding step is diluted in DNAse free water, 10×T4 ligation buffer is added and the mixture kept on ice; ligation final volume is around 150 μL for 10-15 mgs beads. T4 DNA ligase is then added, and the mixture poured over the beads. Reaction is then run at room temperature between 5 to 16 hours.

The DNA tagging step described in the previous paragraph is performed for as many times as there would be variable substituents that are to be connected to a fixed scaffold in the split-and-pool synthesis.

### Example 3: Sorting of Beads

This procedure may e.g. be applied to beads that were used in an assay of the invention, wherein the beads comprise a fluorophore and associated quencher as the probe, and the quencher was removed by the response molecule. After termination of the incubation, the beads are isolated from the incubation media and its response molecule, then the isolated beads (with both fluorescent and non-fluorescent probes) are pooled

The pooled beads were sorted with a commercial fluorescence activated cell sorter (FACS) by gating on the desired property of the beads (either fluorescent or not, and potential other gates for quality control). The positively sorted beads were subjected to DNA-amplification and sequencing.

### Example 4: Bead-Based Secreted Embryonal Alkaline Phosphatase (SEAP) Reporter Assay

Step 1: Preparation of Quenched-Fluorescein Labeled Beads

A quenched fluorescein derivative (such as 1-oxo-3′,6′-diphosphonooxy-spiro[isobenzofuran-3,9′-xanthene]-5-carboxylic acid or 1-oxo-3′,6′-bis(phosphonooxymethoxy)spiro[isobenzofuran-3,9′-xanthene]-5-carboxylic acid) is incubated in aqueous buffer with single-stranded DNA modified with 5′-Amino C6 and a water compatible acylating reagent such as DMT-MM (4-(4,6-dimethoxy-1,3,5-triazin-2-yl)-4-methyl-morpholinium chloride). The DNA encoded library beads, which possess bead-bound DNA barcodes, contains a single-stranded DNA terminus, whose sequence complements that of the above described fluorescein single-stranded DNA conjugate. The fluorescein single-stranded DNA conjugate, upon incubation with a pool of library beads, is annealed with its complement on the beads, to form a stable double-stranded DNA duplex.

Alternatively, the fluorescein single-stranded DNA conjugate may be annealed with another single-stranded DNA oligomer, forming a duplex with overhangs. In this case, the DNA encoded library beads possessing bead-bound DNA barcodes will terminate with a short overhang of 2-4 bases, and this overhang will complement that of the above described fluorescein conjugated double-stranded DNA. The double-stranded DNA oligomer and the barcode will then be covalently joined employing ligase as previously described (see Clark et. al.). Using either of the above methods, library beads may be labeled with any probe, on demand.

Step 2: Concapsulation of Beads and Eukaryotic (THP1-Dual™ KI-hSTING-S154, InvivoGen) Cells and Assay

Using a flow focusing microfluidic chip, aqueous droplets are generated in oil to yield an emulsion of monodisperse droplets. The aqueous phase contains the desired eukaryotic cells, fully functionalized and barcoded beads (additionally labeled with phosphorylated fluorescein) as prepared above, and additives, such that each droplet contains 0, 1, or several of the beads and 0, 1, several, or up to hundreds of eukaryotic cells. Note that additives may include 2′3′-cGAMP in the case of THP1-Dual™ KI-hSTING-S154 cells. Additionally, a positive control (the known irreversible small molecule inhibitor H-151, InvivoGen) may be also be employed as an additive to provide a baseline for later statistical analysis and bead sorting. Droplets are collected in a small reaction tube. Droplets with beads are exposed to several minutes of UV-light to liberate library members from all the beads by photolytic cleavage of the above photolabile linker. The droplets are incubated several hours. The droplets are then heat inactivated or the emulsion is broken by organic solvent, which also stops the detection reaction. Beads are collected on a frit and pooled, and the hit beads having low fluorescence are sorted out of the pool by FACS. The beads with a low fluorescence are then amplified by PCR and sequenced; low fluorescence indicates that the released small-molecule associated to that barcode DNA was successful in inhibiting STING activation and thereby preventing the release of SEAP.

1. A method for screening a DNA-encoded library of chemical structures (2) for activity in a cellular target (11); wherein the cellular target (11) is known to either release, or alter the release, of a response molecule (12) when being contacted with an active chemical structure; wherein the chemical structures (2) of the library, the corresponding encoding DNA (4) and, optionally, a chemical probe (7/8/9) susceptible to the response molecule (12) are covalently linked to beads (1), wherein each bead (1) comprises
a) multiple instances of one sole chemical structure (2) of the library, each instance being covalently linked over a structure linker (3) to the bead (1), the structure linker (3) being cleavable at a cleavable structure linker site (3a); and
b) multiple instances of a DNA sequence (4) encoding for that chemical structure (2), each DNA sequence (4) being covalently linked over a tag linker (5) to the bead (1), the tag linker (5) comprising a cleavable tag linker site (5a) and being cleavable by a cleaving agent (6);
wherein the cleavable structure linker site (3a) is not cleavable under reaction conditions that cleave the cleavable tag linker site (5a), and vice versa;
and the tag linkers (5) and/or the cleavable tag linker sites (5a) and/or the DNA sequences (4) are optionally cleavable by the response molecule (12); provided that, if the encoding DNA sequences (4) and/or the cleavable tag linker sites (5a) and/or the tag linkers (5) are cleavable by the response molecule (12), then the bead (1) is preferably devoid of the response molecule-susceptible chemical probe (7/8/9);
the method comprising the steps of:
(i) either
(i-a) providing, for each individual chemical structure (2) and each individual cellular target (11) to be assayed, an incubation medium (13) comprising the cellular target (11) and one or more bead(s) (1) as defined above, the bead(s) having that individual chemical structure linked thereto, releasing the chemical structures (2) from the bead(s) (1) in the incubation medium (13) by cleaving the structure linkers (3) at the cleavable structure linker site (3a), and incubating the cellular target (11) and the released chemical structures (2) in the incubation medium;
or
(i-b) providing one sole incubation medium (13) comprising the cellular target (11) and all beads (1) as defined above, having all chemical structures (2) of the library linked thereto, separating from the incubation medium aliquots thereof comprising one or more beads (1), releasing in each of the aliquots the chemical structures (2) from the contained bead (1) by cleaving the structure linkers (3) at the cleavable structure linker site (3a), and incubating the cellular target (11) and the released chemical structures (2) in the aliquots of incubation medium (13);
(ii) either, if the encoding DNA sequences (4) and/or the cleavable tag linker sites (5a) and/or the tag linkers (5) are cleavable by the response molecule (12):
(ii-a-1) the incubation media (13) or the aliquots thereof are monitored for release of any encoding DNA sequences (4) or fragments thereof from any beads (1); and if so, all beads (1) are isolated from all incubation media (13) or from all aliquots thereof, and all isolated beads (1) are pooled;
(ii-a-2) the cleavable tag linker sites (5a) in the pooled beads (1) are cleaved by the cleaving agent (6) to release any encoding DNA sequences (4) or fragments thereof;
(ii-a-3) the released encoding DNA sequences (4) or fragments thereof are amplified and sequenced, to identify among them any complete DNA sequences of the DNA encoded library; and
(ii-a-4) the remainder of the complete DNA sequences of the DNA encoded library that were not identified in step (ii-a-3) are correlated with the corresponding chemical structures (2) of the the DNA encoded library;
or alternatively, if the bead(s) (1) comprise(s) the response molecule-susceptible chemical probe (7/8/9):
(ii-b-1) the incubation media (13) or aliquots thereof are monitored for any reaction, or change of reaction, of any probe(s) (7/8/9) to the response molecule (12), and if so, all beads (1) are isolated from all incubation media (13) or from all aliquots thereof and are pooled;
(ii-b-2) beads (1) showing said probe reaction, or said change of probe reaction, are extracted from the pool;
(ii-b-3) the cleavable tag linker sites (5a) in the beads (1) extracted from the pool are cleaved by the cleaving agent (6) to release any DNA sequences (4) covalently linked to the isolated beads (1);
(ii-b-4) the released DNA sequences (4) are amplified and sequenced; and
(ii-b-5) any DNA sequences sequenced in step (ii-b-3) are correlated with corresponding chemical structure(s) (2) of the the DNA encoded library;
and
(iii) any chemical structure (2) so correlated in step (ii-a-4) or (ii-b-5) is selected as a further said active chemical structure.

2. The method of claim 1, wherein the incubation media (13) or the aliquots of the incubation medium (13) thereof have an average bead population λ, defined as
$$λ = \frac{{\sum }_{i = 1}^{M} {( {k}_{m} )}_{i}}{M} \qquad ( 2 )$$
wherein (kₘ)ᵢ is an integer number designating the number of beads in the i-th incubation medium or i-th aliquot of the incubation medium; M is the number of the incubation media or of the aliquots of the incubation medium, respectively; and the sum runs over all M incubation media or over all M aliquots of the incubation medium, respectively; of about 1.0.

3. The method of claim 1, wherein the tag linkers (5) and/or the cleavable tag linker sites (5a) and/or the DNA sequences (4) are cleavable by the response molecule (12); and steps (ii-a-1), (ii-a-2), (ii-a-3) and (ii-a-4) are carried out.

4. The method of claim 3, wherein the cellular target (11) is a prokaryotic cell, in particular a bacterium, known to undergo cell death when contacted with an active chemical structure, and to thereby release a nuclease as the response molecule (12); and the tag linkers (5) and/or the cleavable tag linker sites (5a) and/or the DNA sequences (4) are cleavable by said nuclease.

5. The method of claim 1, wherein the bead(s) (1) comprise(s) a chemical probe (7/8/9) being susceptible to the response molecule (12) and being covalently linked to the bead(s) (1); and steps (ii-b-1), (ii-b-2), (ii-b-3), (ii-b-4) and (ii-b-5) are carried out.

6. The method of claim 5, wherein the cellular target (11) is an eukaryotic cell, modified such that when the desired cellular target or pathway is contacted with a suited chemical structure (2) the response molecule (12) is secreted embryonic alkaline phasphatase (SEAP); and the chemical probe is susceptible to secreted embryonic alkaline phosphatase.

7. The method of claim 5, wherein the chemical probe (7/8/9) is a combination of a fluorophore (7) and a quencher (8) acting by fluorescence resonance energy transfer (FRET) or by contact quenching onto the fluorophore (7); wherein the fluorophore (7) and the quencher (8) are linked to each other over a spacer (9) which is cleavable by the response molecule (12); and wherein in step (ii-b-1) the incubation medium (13) or the aliquots thereof are monitored for cleavage of the spacers (9) by the response molecule (12) by incident fluorescence of the fluorophores (7).

8. The method of claim 1, wherein the cleavable tag linker sites (5a) are nucleotide sequences cleavable by a restriction endonuclease as the cleaving agent (6).

9. The method of claim 8, wherein the tag linker (5) comprises a divalent spacer (5b) of the structure —O—(CH2-CH2-O)ₙ— immediately adjacent to the cleavable tag linker site (5a), wherein n is an integer from 5 to 10, and preferably is 8.

10. The method of claim 1, wherein each bead (1) comprises the multiple instances of the chemical structure (2) linked to the bead (1) over a UV light-cleavable structure linker (3).

11. A bead (1) comprising:
a) a bead core of an organic polymer, in particular polystyrene;
b) multiple instances of one sole chemical structure (2), each instance being covalently linked to the bead over a cleavable structure linker (3) being cleavable at a cleavable structure linker site (3a),
c) multiple instances of a DNA sequence (4) encoding for that sole chemical structure (2), each DNA sequence (4) being covalently linked to the bead (1) over a tag linker (5) containing a cleavable tag linker site (5a) being cleavable by a cleaving agent (6); and the tag linkers (5) and/or the cleavable tag linker sites (5a) and/or the DNA sequences (4) being cleavable by a response molecule (12); wherein the cleavable structure linker site (3a) is not cleavable under reaction conditions that cleave the cleavable tag linker site (5a), and vice versa;
the bead (1) being preferably devoid of any other chemical moiety susceptible, cleavable and/or reactive to that response molecule (12);
or the bead (1) consisting of a), b) and c).

12. The bead of claim 11, wherein the response molecule (12) is a nuclease.

13. The bead (1) of claim 11, wherein the cleavable tag linkers (5) contain as the cleavable moiety (5a) a nucleotide sequence that is cleavable by a restriction endonuclease as the cleaving agent (6).

14. The bead (1) of claim 13, wherein the tag linker (5) comprises a divalent spacer (5b) of the structure —O—(CH2-CH2-O)n- immediately adjacent to the cleavable tag linker site (5a), and wherein n is an integer from 5 to 10, and preferably is 8.

15. The bead (1) of claim 11, wherein the cleavable structure linker site (3a) is cleavable by UV light.


SEQUENCE LISTING (3 sequences)

SEQ ID NO: 1 - protein, 7 aa, Artificial Sequence
  synthetic linker peptide substructure
ENLYFQG

SEQ ID NO: 2 - DNA, 20 bp, Artificial sequence
  Synthetic linker DNA cleaving sequence
gtaacgatccagctgtcact

SEQ ID NO: 3 - protein, 10 aa, Artificial sequence
  Synthetic linker cleaving site
KGDEVDGSGK